(19) United States
(12) Patent Application Publication
(10) Pub. No.: US 2009/0318414 A1
(43) Pub. Date: Dec. 24, 2009

(54) Mono and Combination Therapy with M1/M4 Muscarinic Agonist (Sabcomeline) for Treatment of Prodromal Syndrome

(76) Inventors: Paul Christopher Sharpe, Essex (GB); Peter Robin Blower, Essex (GB); Jill Galloway Chisnall Rasmussen, Essex (GB)

Correspondence Address: NIXON & VANDERHYE, PC, 901 NORTH GLEBE ROAD, 11TH FLOOR, ARLINGTON, VA, 22203, US

(21) Appl. No.: 12/226,423

(22) PCT Filed: Apr. 23, 2007
(86) PCT No.: PCT/GB2007/001463

§ 371 Date: Jan. 16, 2009

(30) Foreign Application Priority Data
Apr. 21, 2006 (GB) 0607952.9

(51) Int. Cl. A61K 31/554; A61P 25/24

(52) U.S. Cl. 514/211.13

(57) ABSTRACT

The invention relates to the use of a functional muscarinic M1/M4 agonist or a pharmaceutically acceptable salt thereof to treat prodromal syndrome and in the manufacture of a medicament for the treatment of prodromal syndrome, and to a method of treatment of prodromal syndrome using a functional muscarinic M1/M4 agonist or a pharmaceutically acceptable salt thereof. It also relates to the use of a pharmaceutical composition comprising a functional muscarinic M1/M4 agonist or a pharmaceutically acceptable salt thereof, and at least one cholinergic agent and/or at least one non-cholinergic agent for the treatment of prodromal syndrome, and to a method of treatment of prodromal syndrome by administration of a such a pharmaceutical composition.

The present invention relates to therapy for the treatment of prodromal syndrome and to a method of treatment of prodromal syndrome.

U.S. Pat. No. 5,278,170 describes a class of compounds which enhance cholinergic neuronal activity via functional action at muscarinic M1/M4 receptors within the central nervous system. A particularly preferred compound from within the scope of this disclosure has been given the common name sabcomeline. The chemical name for sabcomeline is R-(Z)-(-methoxyimino)-(-(1-azabicyclo[2.2.2]oct-3-yl)acetonitrile. For therapeutic administration, it is preferably used in the form of a pharmaceutically acceptable salt, typically the hydrochloride salt, but alternative salts of sabcomeline with pharmaceutically acceptable acids may also be utilised in therapeutic administration, for example salts derived from sabcomeline free base and acids including, but not limited to, hydrobromic acid, phosphoric acid, acetic acid, fumaric acid, maleic acid, salicylic acid, citric acid, lactic acid, oxalic acid and p-toluene sulphonic acid.

Sabcomeline was initially evaluated for its use in the treatment of Alzheimer's disease. Subsequently, a number of documents have disclosed the use of sabcomeline for treating psychotic disorders, for example WO 98/46226. WO 02/03684 further discloses the treatment of psychotic disorders by administration of a muscarinic agonist in combination with a typical or an atypical antipsychotic. Although sabcomeline is disclosed in WO 02/03684 as one of a number of muscarinic agonists suitable for combination with a large number of typical and atypical antipsychotics, exemplification is limited to just one muscarinic agonist (xanomeline) in combination with a small number of antipsychotics, and no specific information or data are recorded concerning combination therapy involving sabcomeline.

It has now been found that functional muscarinic M1/M4 agonists such as sabcomeline or a pharmaceutically acceptable salt thereof may be used advantageously to treat prodromal syndrome.

Prodromal syndrome is defined as an impairment in global functioning resulting from a constellation of symptoms indicating that a patient is at risk of developing a psychosis or psychotic disease

These symptoms include but are not limited to: affective symptoms such as anxiety, apathy, agitation, anger or irritability, depressed mood, sleep disturbance; cognitive impairment such as poor concentration, disturbance in attention and/or memory; change in usual behaviour including, social withdrawal, loss of interest in work and hobbies, deterioration of hygiene and grooming. These symptoms may vary from patient to patient and may lead to the development of a psychotic disease, episode, disorder or breakdown but this is not necessarily the inevitable outcome.

For the avoidance of doubt, it is intended that the term psychotic disease covers the full spectrum of psychotic disorders known to the skilled person. These include, but are not limited to, the following psychotic disorders: bi-polar disorder, schizophrenia, including, catatonic, disorganised, paranoid, residual and undifferentiated schizophrenia; schizophreniform disorder; schizoaffective disorder; delusional disorder; brief psychotic disorder; shared psychotic disorder; psychotic disorder due to a general medical condition.

In a first aspect therefore, the invention provides a method of treatment of prodromal syndrome by administration of a functional muscarinic M1/M4 agonist or a pharmaceutically acceptable salt thereof.

In a further aspect, the invention provides the use of a functional muscarinic M1/M4 agonist or a pharmaceutically acceptable salt thereof in the manufacture of a medicament for the treatment of prodromal syndrome.

The invention also provides the use of a functional muscarinic M1/M4 agonist or a pharmaceutically acceptable salt thereof for the treatment of prodromal syndrome.

The invention further provides a functional muscarinic M1/M4 agonist or a pharmaceutically acceptable salt thereof for use in the treatment of prodromal syndrome.

The invention further provides
a) a method of treatment by administration of a functional muscarinic M1/M4 agonist, or a pharmaceutically acceptable salt thereof, of;
b) the use of a functional muscarinic M1/M4 agonist or a pharmaceutically acceptable salt thereof in the manufacture of a medicament for the treatment of; and
c) the use of a functional muscarinic M1/M4 agonist or a pharmaceutically acceptable salt thereof for the treatment of
i) affective symptoms, cognitive impairment and/or change in usual behaviour;
ii) anxiety, apathy, agitation, anger or irritability, depressed mood, sleep disturbance, poor concentration, disturbance in attention and/or memory, social withdrawal, loss of interest in work and hobbies, and/or deterioration of hygiene and grooming;
iii) anxiety, apathy, agitation, anger or irritability, depressed mood, sleep disturbance, social withdrawal, loss of interest in work and hobbies, and/or deterioration of hygiene and grooming;
iv) anxiety, apathy, agitation, anger or irritability, depressed mood and/or sleep disturbance;
v) poor concentration and/or disturbance in attention and/or memory;
vi) social withdrawal, loss of interest in work and hobbies and/or deterioration of hygiene and grooming;
vii) apathy, agitation, anger or irritability, depressed mood, sleep disturbance, poor concentration, disturbance in attention and/or memory, social withdrawal, loss of interest in work and hobbies, and/or deterioration of hygiene and grooming; or
viii) apathy, agitation, anger or irritability, depressed mood and/or sleep disturbance.

Functional muscarinic M1/M4 agonists are compounds which enhance cholinergic neuronal activity at the muscarinic M1/M4 receptors predominantly. This functional selectivity results in a level of safety and tolerability advantageous for use in the treatment of prodromal syndrome. Sabcomeline is one such functional muscarinic M1/M4 agonist. Other suitable functional M1/M4 agonists or combinations thereof may also be used.

Preferably, the functional muscarinic M1/M4 agonist or a pharmaceutically acceptable salt thereof, in particular sabcomeline or a pharmaceutically acceptable salt thereof is administered independently of any other medication.

For therapeutic administration according to the present invention, the functional muscarinic M1/M4 agonist or a pharmaceutically acceptable salt thereof, in particular sabcomeline may be employed in the form of its free base, but is preferably used in the form of a pharmaceutically acceptable salt, typically the hydrochloride salt.

Alternative salts of the functional muscarinic M1/M4 agonist, in particular sabcomeline with pharmaceutically acceptable acids may also be utilised in therapeutic administration, for example salts derived from the functional muscarinic M1/M4 agonist free base, in particular sabcomeline free base and acids including, but not limited to, hydrobromic acid, phosphoric acid, acetic acid, fumaric acid, maleic acid, salicylic acid, citric acid, oxalic acid, lactic acid, malic acid, methanesulphonic acid and p-toluene sulphonic acid.

All solvates and all alternative physical forms of the functional muscarinic M1/M4 agonist, in particular sabcomeline or its pharmaceutically acceptable derivatives as described herein, including but not limited to alternative crystalline forms, amorphous forms and polymorphs are also within the scope of this invention, and all references to a functional muscarinic M1/M4 agonist, in particular sabcomeline herein include all pharmaceutically acceptable salts, and all solvates and alternative physical forms thereof.

For therapeutic administration, the functional muscarinic M1/M4 agonist or a pharmaceutically acceptable salt thereof, in particular sabcomeline or its pharmaceutically acceptable salts or solvates may be administered in pure form, but will preferably be formulated into any suitable pharmaceutically acceptable and effective composition which provides effective levels of the active ingredient in the body. The choice of the most appropriate pharmaceutical compositions is within the skill of the art. Suitable formulations include, but are not limited to tablets, capsules, powders, granules, lozenges, suppositories, reconstitutable powders, or liquid preparations such as oral or sterile parenteral solutions or suspensions.

The treatment of prodromal syndrome may include administering a functional muscarinic M1/M4 agonist or a pharmaceutically acceptable salt thereof, in particular sabcomeline or a pharmaceutically acceptable salt thereof, at a dose of between 10 μg-200 μg. Preferably, the dose is between 20 μg-100 μg. More preferably, the dose is between 25 μg-50 μg.

The dose may be administered as a single dose or twice daily. Ideally, the functional muscarinic M1/M4 agonist or a pharmaceutically acceptable salt thereof, in particular sabcomeline or a pharmaceutically acceptable salt thereof, is administered at a dose of 25 μg twice daily.

Typically, the functional muscarinic M1/M4 agonist or a pharmaceutically acceptable salt thereof, in particular sabcomeline or a pharmaceutically acceptable salt thereof, is administered to the patient at dose ranges of 20 to 50 μg total daily dose with titration to optimal dose in the range 10 to 200 μg total daily dose.

In order to obtain consistency of administration it is preferred that the compositions are in the form of a unit dose.

Such unit dose presentation forms for oral administration may be in the form of solid oral compositions, such as tablets and capsules, and may contain conventional excipients such as

binding agents, for example syrup, acacia, gelatin, sorbitol, tragacanth, or polyvinylpyrrolidone;

fillers, for example lactose, sugar, maize-starch, calcium phosphate, sorbitol or glycine;

tabletting lubricants, for example magnesium stearate; disintegrants, for example starch, polyvinylpyrrolidone, sodium starch glycollate or microcrystalline cellulose; and

pharmaceutically acceptable wetting agents such as sodium lauryl sulphate.

Solid oral compositions may be prepared by conventional methods of blending, filling, tabletting or the like. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations for use in the invention may be in the form of, for example, emulsions, syrups, suspensions or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, or hydrogenated edible fats;

emulsifying agents, for example lecithin, sorbitan monooleate, or acacia;

non-aqueous vehicles (which may include edible oils), for example almond oil, fractionated coconut oil, oily esters such as esters of glycerine, propylene glycol, or ethyl alcohol;

preservatives, for example methyl or propyl p-hydroxybenzoate or sorbic acid; and

if desired, conventional flavouring or colouring agents.

For parenteral administration (for example intravenous, intravascular or subcutaneous administration), fluid unit dosage forms are prepared utilizing the component or the combination of the components and a sterile vehicle, and, depending on the concentration used, can be either suspended or dissolved in the vehicle.

In preparing solutions the component(s) can be dissolved in water for injection and filter sterilized before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum.

Parenteral suspensions are prepared in substantially the same manner, except that the component is suspended in the vehicle instead of being dissolved, and sterilization cannot be accomplished by filtration. The component(s) can be sterilized by exposure to ethylene oxide before suspending in the sterile vehicle. Advantageously, a surfactant or wetting agent is included in the composition to facilitate uniform distribution of the component or the combination of the components.

Alternatively, the components may be prepared in solid form which melts on contact with the tongue of the patient, for example in the form of orally disintegrating tablets sold under the trade name ZYDIS®.

The compositions of the invention may also be formulated as depot preparations.

Such long acting formulations may be administered by implantation (for example subcutaneously or intramuscularly) or by intramuscular injection. Thus, for example, the components of the invention may be formulated with suitable polymeric or hydrophobic materials (for example as an emulsion in an acceptable oil) or ion exchange resins, or as sparingly soluble derivatives, for example, as a sparingly soluble salt.

The compositions of the invention may contain from 0.1% to 99% by weight, preferably from 10-60% by weight, of the active material, depending on the method of administration.

The unit dose of the functional muscarinic M1/M4 agonist or a pharmaceutically acceptable salt thereof, in particular sabcomeline or a pharmaceutically acceptable salt thereof, is in the range of 10 μg-200 μg. Preferably, the dose is between 20 μg-100 μg. More preferably, the dose is between 25 μg-50 μg. The dose may be administered as a single dose or twice daily. Ideally, the functional muscarinic M1/M4 agonist or a pharmaceutically acceptable salt thereof, in particular sabcomeline or a pharmaceutically acceptable salt thereof, is administered at a dose of 25 μg twice daily.

It has also been found that a functional muscarinic M1/M4 agonist or a pharmaceutically acceptable salt thereof, in particular sabcomeline or a pharmaceutically acceptable salt thereof may advantageously be administered in combination with at least one agent for “add-on therapy”. This combination provides improved treatment of prodromal syndrome.

The agent for add-on therapy may be an agent for augmenting cholinergic activity (hereinafter referred to as a cholinergic agent) such as an atypical anti-psychotic agent, or nicotinic agonist or a 5HT6 antagonist; or an agent which provides benefit other than via a cholinergic mechanism (hereinafter referred to as a non-cholinergic agent) such as a neuroprotective agent, a neuroleptic agent, atypical antipsychotic, anti-depressant, anxiolytic or mood stabiliser.

These combinations, and corresponding uses and methods of treatment of the invention may also provide advantages in treatment of patients who fail to respond adequately or who are resistant to other known treatments.

In a further aspect, therefore, the invention provides a pharmaceutical composition comprising a functional muscarinic M1/M4 agonist or a pharmaceutically acceptable salt thereof, in particular sabcomeline or a pharmaceutically acceptable salt thereof and

at least one cholinergic agent and/or

at least one non-cholinergic agent.

The invention also provides the use of a pharmaceutical composition comprising a functional muscarinic M1/M4 agonist or a pharmaceutically acceptable salt thereof, in particular sabcomeline or a pharmaceutically acceptable salt thereof and at least one cholinergic agent and/or at least one non-cholinergic agent for the treatment of prodromal syndrome.

The combination therapies of the invention are preferably administered adjunctively. By adjunctive administration is meant the coterminous or overlapping administration of each of the components in the form of separate pharmaceutical compositions or devices. Any and all treatment regimes in which a patient receives separate but coterminous or overlapping therapeutic administration of a functional muscarinic M1/M4 agonist or a pharmaceutically acceptable salt thereof, in particular sabcomeline or a pharmaceutically acceptable salt thereof and at least one cholinergic agent and/or at least one non-cholinergic agent are within the scope of the current invention.

In one embodiment of adjunctive therapeutic administration as described herein, a patient is typically stabilised on a therapeutic administration of one or more of the components for a period of time and then receives administration of another component.

Within the scope of this invention, it is preferred that a functional muscarinic M1/M4 agonist or a pharmaceutically acceptable salt thereof, in particular sabcomeline or a pharmaceutically acceptable salt thereof is administered as adjunctive therapeutic treatment to patients who are receiving administration of at least one cholinergic agent and/or at least one non-cholinergic agent.

However, the scope of the invention also includes the adjunctive therapeutic administration of at least one cholinergic agent and/or at least one non-cholinergic agent to patients who are receiving administration of a functional muscarinic M1/M4 agonist or a pharmaceutically acceptable salt thereof.

The latter is in particular sabcomeline or a pharmaceutically acceptable salt thereof.

The combination therapies of the invention may also be administered simultaneously. By simultaneous administration is meant a treatment regime wherein the individual components are administered together, either in the form of a single pharmaceutical composition or device comprising or containing two or more components, or as separate compositions or devices, each comprising one of the components, administered simultaneously. Such combinations of the separate individual components for simultaneous combination may be provided in the form of a kit-of-parts.

A neuroprotective agent may be defined as a compound which is intended to help limit the damage suffered by a nerve or neural tissue such as, for example, spinal cord, brain or nerve, when the blood supply is cut off or there is a traumatic injury.

It is envisaged that psychotic disorders or diseases may be due in part to the breakdown of neurons or nerve ends such as to cause a breakdown of neural integrity. It is believed that neuroprotective agents help to prevent or stop the breakdown of neurons and neural integrity. Administering a neuroprotective agent alters the underlying pathology affecting integrity of neural function.

Neuroprotective agents include, but are not limited to, some types of antioxidants, anti-inflammatories and anti-psychotics such as lithium. For example, neuroprotective agents include, but are not limited to, anti-oxidants, for example Vitamin E, eicosapentaenoic acid (EPA), docosahexaenoic acid (DHA), and anti-inflammatories such as non-steroidal anti-inflammatory, cyclo-oxygenase-2 (cox-2) inhibitors and statins.

The term neuroleptic or atypical antipsychotic refers to drugs which have the effects on cognition and behaviour of antipsychotic drugs that reduce confusion, delusions, hallucinations, and psychomotor agitation in patients with psychoses.

Also known as major tranquilizers and antipsychotic drugs, neuroleptic agents include, but are not limited to: phenothiazines, further divided into the aliphatics, piperidines, and piperazines, thioxanthenes (e.g., droperidol), butyrophenones (e.g., haloperidol), dibenzoxazepines (e.g., loxapine), dihydroindolone (e.g., molindone), diphenylbutylpiperidine (e.g., pimozide), benzisoxazole (e.g., risperidone).

Preferably, the functional muscarinic M1/M4 agonist or a pharmaceutically acceptable salt thereof, in particular sabcomeline or a pharmaceutically acceptable salt thereof, cholinergic agent and/or non-cholinergic agent are present in the ranges 1-100%, 0.0-99% and 0.0-99% respectively.

For example, the functional muscarinic M1/M4 agonist or a pharmaceutically acceptable salt thereof, in particular sabcomeline or a pharmaceutically acceptable salt thereof, cholinergic agent and/or non-cholinergic agent, maybe administered, by weight, in the ranges 10 μg-200 μg, 0.05 μg, and 0.0-5 μg respectively.

Preferably, the functional muscarinic M1/M4 agonist or a pharmaceutically acceptable salt thereof, in particular sabcomeline or a pharmaceutically acceptable salt thereof, is present in the range between 20 μg-100 μg.

More preferably, the functional muscarinic M1/M4 agonist or a pharmaceutically acceptable salt thereof, in particular sabcomeline or a pharmaceutically acceptable salt thereof, is present in the range between 25 μg-50 μg.

When a chosen neuroprotective agent is also an antipsychotic, it is believed that the clinical utility of the combination of the functional muscarinic M1/M4 agonist or a pharmaceutically acceptable salt thereof, in particular sabcomeline or a pharmaceutically acceptable salt thereof, and antipsychotic may vary between different members of the atypical antipsychotic drug class, depending on their different affinities for various sub-types of neurochemical receptors.

For example, in addition to their affinities for dopamine and serotonin receptors, members of the atypical antipsychotic class may vary in their affinity for muscarinic and histamine receptor sub-types.

The activity of atypical antipsychotics at muscarinic receptor subtypes are such that properties of negligible affinity, weak agonist activity and weak antagonist activity have been reported amongst the atypical antipsychotic drug class.

As an example, the M1/M4 receptor agonist properties of a functional muscarinic M1/M4 agonist, in particular sabcomeline may enhance functional cholinergic activity and, when administered in combination, provide benefit by:
i) enhancing functional cholinergic activity in combination with an atypical antipsychotic that itself has little or no affinity for muscarinic receptors (e.g. risperidone);
ii) providing additive functional cholinergic activity in combination with an atypical antipsychotic drug that has weak muscarinic receptor agonist effects (e.g. clozapine or N-desmethylclozapine);
iii) competing for muscarinic receptors and thereby reducing the anticholinergic functional effects of an atypical antipsychotic drug that possesses muscarinic receptor antagonist properties (e.g. olanzapine).

As well as muscarinic and histaminergic receptors there are other mechanisms that may have benefit or adverse effects on cognition. For instance drugs with 5-HT6 antagonist and an adrenergic (2 antagonist properties may also be of benefit. Some atypical antipsychotics also have these benefits.

For adjunctive or simultaneous therapeutic administration according to the combination therapies of the invention, the functional muscarinic M1/M4 agonist or a pharmaceutically acceptable salt thereof, in particular sabcomeline or its pharmaceutically acceptable salts and the cholinergic agent and/or non-cholinergic agent or their pharmaceutically acceptable salts, derivatives or solvates may each be administered in pure form.

However, each of the components will preferably be formulated into any suitable pharmaceutically acceptable and effective composition which provides effective levels of the respective component in the body.

For the combination therapies, the daily and unit doses of the cholinergic agent and/or non-cholinergic agent will depend upon which cholinergic agent and/or non-cholinergic agent is employed, but may typically be the recommended or approved dosage for the specific cholinergic agent and/or non-cholinergic agent when administered as monotherapy.

In a preferred aspect of the invention, adjunctive administration of a functional muscarinic M1/M4 agonist or a pharmaceutically acceptable salt thereof, in particular sabcomeline or a pharmaceutically acceptable salt thereof may permit lower doses of the cholinergic agent and/or non-cholinergic agent than those normally recommended when the cholinergic agent and/or non-cholinergic agent is prescribed as monotherapy.

The following are given by way of example only to illustrate and aid understanding of the invention:

### EXAMPLE 1

An example of a method of preparation of sabcomeline is as follows: to a stirred solution of potassium tert.-butoxide (94.1 g; 0.84 mol) in tetrahydrofuran (250 ml) under nitrogen is added a solution of 3-(cyanomethyl)quinucidine (60 g; 0.4 mol) in tetrahydrofuran (150 ml) during a period of 10 min.

The reaction is stirred for 10 minutes then cooled to 0øC. Isoamyl nitrite (51.5 g 0.44 mol) is added at a rate such that the internal temperature does not exceed 25° C.

The reaction is stirred for 20 minutes then diluted with dimethylsulphoxide (500 ml). Methyl tosylate (134 g; 0.72 mol) is added as a solution in dimethylsulphoxide (100 ml) at a rate such that the temperature does not exceed 35° C.

After a further 20 minutes aqueous potassium carbonate (ca 5wt % 500 ml) is added and the reaction extracted with ethyl acetate (5×200 ml). The ethyl acetate extract is washed with 5 wt % aqueous potassium carbonate (4×250 ml), then saturated potassium carbonate (50 ml).

The combined aqueous layers are re-extracted with ethyl acetate (500 ml) which is washed as above. The combined organic extracts are dried over anhydrous potassium carbonate (200 g) and concentrated in vacuo to give a brown oil containing ca. 80 wt % 3-[(cyano)(methoxyimino)-methyl]quinuclidine as a 4:1 mixture of Z:E isomers, (47.4 g; 0.245 mol; 61%).

### EXAMPLE 2

The following patient study was a small Phase IIa, proof of concept, 51-day, multicentre, double-blind, placebo-controlled, rising dose parallel study of the efficacy and tolerability of sabcomeline in patients with acute exacerbation of chronic schizophrenia. A total of twenty eight patients, nineteen received sabcomeline and nine patients received placebo. Daily doses of sabcomeline were titrated from 50 (g daily through 100 (g to 150 (g daily over nine days.

The Primary Objective of the study was:
To assess the efficacy of sabcomeline in patients with schizophrenia (includes: effects on positive and negative symptoms of schizophrenia and general psychopathology)

A Secondary Objective of the study was:
To assess the effects of sabcomeline on neurocognitive function in patients with schizophrenia
To study the safety and tolerability of sabcomeline in the treatment of patients with schizophrenia.

The results of the study showed the following:
1. No effect on PANSS positive symptoms—delusions, hallucinations
2. A trend in the PANSS negative subscale. A further review of these results indicated that this treatment difference was due to special effects of sabcomeline on symptoms:
   Benefit in PANSS Negative—blunted affect, emotional withdrawal, difficulty in abstract thinking, lack of spontaneity, stereotypical thinking
   No benefit in PANSS Negative—poor rapport, passive/apathetic social withdrawal
3. A trend in the PANSS general psychopathology subscale. A further review of these results indicated that this treatment difference was due to special effects of sabcomeline on symptoms such as anxiety, depression, motor retardation, unusual thought content, and lack of judgement and insight.
   Benefit seen in PANSS General Psychopath—guilt feelings, tension, mannerism/posturing, motor retardation, poor attention, poor volition, pre-occupation, anxiety, depression, active social avoidance.
   No benefit seen in PANSS General Psychopath—unusual thought content, lack of judgment/insight,
   Unanalysable:
   PANSS General Psychopathology - somatic concern, uncooperativeness, disorientation, poor impulse control
4. A trend in verbal memory (one area of the Rivermead Story test), per protocol analysis.
5. A trend in selective attention and flexibility of thinking (one area of the Stroop Colour Test), per protocol analysis.
6. A trend in attention and speed of processing (one area of Trail Making test B), per protocol analysis

This study was not powered to show statistical significance but rather to generate hypotheses about the potential efficacy of sabcomeline in the treatment of the positive, negative and cognitive symptoms of schizophrenia.

Analysis of the data for 8 severe patients and 4 placebo set out below demonstrated that sabcomeline had benefit in the treatment of affective symptoms; cognitive impairment and change in behaviour, indicating potential benefit in the treatment of the constellation of symptoms defining prodromal syndrome.

TABLE
Positive and Negative syndrome Scale (PANSS) Item analysis on
each patient with at least moderate severity ((4) at baseline

| | Sabcomeline | | Placebo | |
|---|---|---|---|---|
| | Total N = 8 | | Total N = 4 | |
| PANSS | Baseline | Endpoint | Baseline | Endpoint |
| Positive Items | | | | |
| Delusions | 8 | 2/8 | 4 | 3/4 |
| Hallucinations | 4 | 2/4 | 4 | 4/4 |
| Negative Items | | | | |
| Blunted Affect | 8 | 7/8 | 0 | 2* |
| Emotional Withdrawal | 7 | 7/7 | 1 | 1/1 |
| Poor rapport | 4 | 3/4 | 2 | 2/2 |
| Passive/Apathetic | 6 | 2/6 | 3 | 0/3 |
| Social Withdrawal | | | | |
| Difficulty in abstract | 6 | 2/6 | 2 | 1/2 |
| thinking | | | | |
| Lack of spontaneity | 6 | 4/6 | 0 | 0 |
| Stereotypical thinking | 3 | 3/3 | 2 | 2/2 |
| General | | | | |
| Psychopathology | | | | |
| Somatic Concern | 0 | 0 | 1 | 0/1 |
| Anxiety | 4 | 3/4 + 1* | 0 | 2* |
| Guilt Feelings | 2 | 2/2 + 1* | 0 | 0 |
| Tension | 6 | 3/6 | 2 | 0/2 |
| Mannerism Posturing | 3 | 3/3* | 2 | 1/2 |
| Depression | 3 | 2/3 | 0 | 0 |
| Motor Retardation | 4 | 4/4 | 0 | 0 |
| Uncooperativeness | 0 | 0 | 1 | 1/1 |
| Unusual Thought | 6 | 2/6 | 4 | 3/4 |
| Content | | | | |
| Disorientation | 0 | 0 | 0 | 0 |
| Poor Attention | 5 | 5/5 | 1 | 0/1 |
| Lack of judgement/ | 4 | 1/4 | 4 | 1/4 |
| insight | | | | |
| Poor volition | 4 | 4/4 | 1 | 0/1 |
| Poor impulse control | 0 | 0 | 0 | 0 |
| Preoccupation | 5 | 3/5 | 1 | 1/1 |
| Active Social Avoidance | 4 | 1/4 | 0 | 0 |

*indicates worsening of symptoms

The safety and tolerability data for sabcomeline in this study are consistent with observations from clinical studies in other indications (see Example 3) which reveal a generally well-tolerated and safe compound.

### EXAMPLE 3

Sabcomeline 50 (g daily, or 25 (g twice daily, has also be evaluated in two 24-week placebo-controlled trials that included 880 patients with Alzheimer's disease. Sabcomeline was safe and well-tolerated across the dose range examined.

It will be appreciated, that sabcomeline is an example of a functional muscarinic M1/M4 agonist which is used in the present invention. Other suitable functional M1/M4 agonists or combinations thereof may also be used.

1-39. (canceled)

40. A method of treatment of prodromal syndrome by administration of a functional muscarinic M1/M4 agonist or a pharmaceutically acceptable salt thereof.

41. The method claimed in claim 40, wherein said treatment is of affective symptoms, cognitive impairment or change in usual behaviour.

42. The method claimed in claim 40, wherein said treatment is of anxiety, apathy, agitation, anger or irritability, depressed mood, sleep disturbance, poor concentration, disturbance in attention and/or memory, social withdrawal, loss of interest in work and hobbies, and/or deterioration of hygiene and grooming.

43. The method claimed in claim 40, wherein said treatment is of anxiety, apathy, agitation, anger or irritability, depressed mood, sleep disturbance, social withdrawal, loss of interest in work and hobbies, and/or deterioration of hygiene and grooming.

44. The method claimed in claim 40, wherein said treatment is of anxiety, apathy, agitation, anger or irritability, depressed mood and/or sleep disturbance.

45. The method claimed in claim 40, wherein said treatment is of poor concentration and/or disturbance in attention and/or memory.

46. The method claimed in claim 40, wherein said treatment is of social withdrawal, loss of interest in work and hobbies and/or deterioration of hygiene and grooming.

47. The method claimed in claim 40, wherein said treatment is of apathy, agitation, anger or irritability, depressed mood, sleep disturbance, poor concentration, disturbance in attention and/or memory, social withdrawal, loss of interest in work and hobbies, and/or deterioration of hygiene and grooming.

48. The method claimed in claim 40, wherein said treatment is of apathy, agitation, anger or irritability, depressed mood and/or sleep disturbance.

49. The method claimed in claim 40, wherein said treatment comprises administering the functional muscarinic M1/M4 agonist Sabcomeline at a dose of between 10 μg-200 μg.

50. The method claimed in claim 40, wherein said treatment comprises administering the functional muscarinic M1/M4 agonist at a dose of between 20 μg-100 μg.

51. The method claimed in claim 40, wherein said treatment comprises administering the functional muscarinic M1/M4 agonist Sabcomeline at a dose of between 25 μg-50 μg.

52. The method claimed in claim 40, wherein said treatment comprises administering the functional muscarinic M1/M4 agonist as a single dose or twice daily.

53. The method claimed in claim 40, wherein said treatment comprises administering the functional muscarinic M1/M4 agonist at a dose of 25 μg twice daily.

54. The method claimed in claim 40, wherein said functional muscarinic M1/M4 agonist is sabcomeline.

55. A pharmaceutical composition comprising as components a functional muscarinic M1/M4 agonist or a pharmaceutically acceptable salt thereof, and at least one cholinergic agent and/or at least one non-cholinergic agent.

56. A pharmaceutical composition as claimed in claim 55, wherein the functional muscarinic M1/M4 agonist or a pharmaceutically acceptable salt thereof is sabcomeline or a pharmaceutically acceptable salt thereof.

57. A method of treatment of prodromal syndrome by administration of a pharmaceutical composition as claimed in claim 55.

58. A method as claimed in claim 57, comprising the coterminous or overlapping administration of each of the components in the form of separate pharmaceutical compositions or devices.

59. A method as claimed in claim 57, wherein a patient is stabilised on a therapeutic administration of one or more of the components for a period of time and then receives administration of one or more other components.

60. A method as claimed in claim 57, comprising the simultaneous administration of the components are administered together, in the form of a single pharmaceutical composition or device comprising or containing two or more components, or as separate compositions or devices, each comprising one of the components.
